(19) United States
(12) Patent Application Publication
(10) Pub. No.: US 2016/0270686 A1
(43) Pub. Date: Sep. 22, 2016

(54) SYSTEM AND METHOD FOR MAGNETIC ASSESSMENT OF BODY IRON STORES

(71) Applicant: THE TRUSTEES OF DARTMOUTH COLLEGE, Hanover, NH (US)

(72) Inventors: Solomon G. Diamond, Hanover, NH (US); Bradley W. Ficko, West Lebanon, NH (US)

(21) Appl. No.: 15/167,773

(22) Filed: May 27, 2016

Related U.S. Application Data
(63) Continuation-in-part of application No. PCT/US15/61851, filed on Nov. 20, 2015.
Application No. PCT/US15/61851 is a continuation-in-part of application No. PCT/US15/29481, filed on May 6, 2015.
Application No. PCT/US15/29481 is a continuation-in-part of application No. 14/420,828, filed on Feb. 10, 2015, now Pat. No. 9,395,425.
(60) Provisional application No. 62/082,459, filed on Nov. 20, 2014.
(60) Provisional application No. 61/989,986, filed on May 7, 2014.
(60) Provisional application No. 61/693,044, filed on Aug. 24, 2012.

(51) Int. Cl. A61B 5/05; A61B 5/145; A61B 5/00; G01R 33/16

(52) U.S. Cl. CPC A61B 5/05; G01R 33/16; A61B 5/14546; A61B 5/4504; A61B 5/4244; A61B 2562/0223

(57) ABSTRACT

A system for magnetic assessment of body iron stores includes excitation coils adapted to generate multiple-frequency alternating current (AC) magnetic fields and to partially magnetically saturate iron. The system further includes one or more detection coils adapted to detect the AC magnetic fields. A signal processor uses lock-in amplifiers and linear regression to measures changes to the multiple-frequency AC magnetic fields caused by proximity to iron. A method for magnetic assessment of body iron stores includes generating multiple-frequency AC magnetic fields and detecting changes to the AC magnetic fields caused by proximity to iron. The method further includes partially magnetically saturating iron, thereby generating non-linear responses, harmonic frequencies, and intermodulation frequencies.

### RELATED APPLICATIONS

The present application is a continuation-in-part of PCT application PCT/US15/61851 filed 20 Nov. 2015, which in turn claims priority to U.S. Provisional Patent Application No. 62/082,459 filed 20 Nov. 2014. This application is also a continuation-in-part of PCT Application No. PCT/US 15/29481 filed 6 May 2015, which in turn claims priority to U.S. Provisional Patent Application No. 61/989,986 filed 7 May 2014. This application is also a continuation-in-part of U.S. patent application Ser. No. 14/420,828 filed 10 Feb. 2015, which is a §371 application of PCT Application No. PCT/US 13/56436 filed 23 Aug. 2013. PCT Application No. PCT/US13/56436 in turn claims priority to U.S. Provisional Patent Application No. 61/693,044 filed 24 Aug. 2012. The contents of the aforementioned patent applications are incorporated herein by reference in their entireties.

### BACKGROUND

The invention relates to systems and methods for measuring the level of iron stored in humans Iron levels provide an important measure for disease diagnosis and prognosis. For example, low iron levels cause iron-deficiency anemia, which is endemic in the developing world and prevalent in the developed world. Infants with iron-deficiency anemia have poorer cognitive, motor, social-emotional, and neurophysiological development. Conversely, high iron levels cause iron-overload in adults. Iron-overload poses several health risks such as increased rates of cancer and cardio-vascular disease, and may produce symptoms that mimic other ailments. Hemochromatosis is a genetic disorder of iron storage resulting in excess accumulation of iron in the body (i.e., iron-overload). Currently, no effective way to screen for iron-overload exists. Hemochromatosis is largely underdiagnosed worldwide due to lack of an adequate test, but it is fairly simple to manage once identified.

The gold standard for measuring iron deficiency is bone marrow biopsy with Prussian blue staining. However, due its invasiveness, the procedure is rarely performed. The most widely used measure for iron deficiency or for iron accumulation is the saturation ratio of serum transferrin receptor to serum ferritin in blood. However, serum ferritin is not always reliable because it changes during infections from common conditions such as malaria. Zinc protoporphyrin is a cost effective method, but it can be affected by the concentration of lead in blood and by chronic disease. Non-invasive methods for measuring iron levels include Magnetic Resonance Imaging (MRI) and magnetic measurements using Super Conducting Quantum Interference Devices (SQUIDs). Unfortunately, both of these methods require equipment that is prohibitively bulky and expensive for routine screening in the field.

### SUMMARY

A system for magnetic assessment of body iron stores includes a first excitation coil adapted to generate a magnetic field, a signal generator configured to provide alternating current (AC) signals with a plurality of different frequencies to the first excitation coil thereby generating an AC magnetic field with a plurality of frequencies, one or more detection coils adapted to detect the AC magnetic fields, and a signal processor coupled to the one or more detection coils and adapted to measure changes to the AC magnetic fields caused by proximity of the excitation and detection coils to iron.

A method for magnetic assessment of body iron stores includes generating alternating current (AC) signals with a plurality of different frequencies, applying the AC signals to an excitation coil for generating a plurality of AC magnetic fields at different frequencies, detecting the AC magnetic fields with one or more detection coils, disposing a sample near one or more detection coils wherein iron in the sample causes a change to the AC magnetic fields, and measuring changes to the AC magnetic fields with a signal processor.

### BRIEF DESCRIPTION OF THE FIGURES

FIG. 1 is a schematic diagram showing one embodiment of a system for magnetic assessment of body iron stores.

FIG. 2 is a schematic diagram showing one embodiment of a system for magnetic assessment of body iron stores.

FIG. 3 shows an alternate orientation for excitation and detection coils used in a system for magnetic assessment of body iron stores.

FIG. 4 shows an alternate orientation for excitation and detection coils used in a system for magnetic assessment of body iron stores.

FIG. 5 shows an alternate orientation for excitation and detection coils that includes a permanent magnet, used in a system for magnetic assessment of body iron stores.

FIG. 6 shows an alternate orientation for excitation and detection coils used in a system for magnetic assessment of body iron stores.

FIG. 7 is a block diagram showing one embodiment of a signal processor used in a system for magnetic assessment of body iron stores.

FIG. 8 is a block diagram showing steps of one embodiment of a method for magnetic assessment of body iron stores.

FIG. 9 is a block diagram showing an overview of different types of measurements and their embodiments.

FIG. 10 shows one embodiment with an array of excitation coils configured to form a focal magnetic field.

FIG. 11 shows an exemplary line scan from an embodiment of a method for magnetic assessment of body iron stores.

FIG. 12 shows an iron sensitivity plot calculated from a line scan depicted above a cross-sectional representation of a chest, in an embodiment.

FIG. 13 is a block diagram showing steps of one embodiment of a method for magnetic assessment of body iron stores based on spectroscopic measurements.

FIG. 14A shows a plot of magnetic susceptibility versus time for a series of measurements performed at low and intermediate frequency AC magnetic fields.

FIG. 14B shows a ratio of the low and high frequency measurements of FIG. 14A.

FIG. 15 shows exemplary results from one embodiment of a method for magnetic assessment of body iron stores compared to mass spectrometry measurements.

FIG. 16 shows one embodiment of harmonic and intermodulation frequencies resulting from nonlinear measurements.

FIG. 17 is a block diagram showing steps of one embodiment of a method for magnetic assessment of body iron stores based on nonlinear measurements.

### DETAILED DESCRIPTION OF THE EMBODIMENTS

The system and method for magnetic assessment of body iron stores disclosed herein includes a simple-to-use, potentially low-cost, point-of-care portable electronic device for noninvasive magnetic assessment of iron in bone marrow. The device measures biological iron content using alternating current (AC) magnetic susceptibility. Studies of bone marrow have mapped the percentage of red and yellow marrow over time in different bone structures. Red marrow is important for iron assessment because the red marrow contains up to 60 percent hematopoietic cells containing iron in different forms. As a person ages, red marrow in bone may be converted to yellow marrow but not at equal rates for all bones. The sternum and vertebra typically retain a higher percentage of red marrow than bones like the tibia and femur. For pediatric patients, the sternum and vertebra typically contain primarily red marrow before 5 years of age. In adults, the sternum and vertebra contain between 50 to 75 percent red marrow.

When a magnetically susceptible material is subjected to an external magnetic field H, a resulting magnetic induction or B-field is B=μ₀(H+M), where po is magnetic permeability in a vacuum, H is an externally applied magnetic field strength, and M is a magnetization field that arises from the magnetically susceptible material. In an AC magnetic field, susceptibility is frequency dependent and has in-phase and out-of-phase components. Although M-field only exists inside of a magnetic material, it gives rise to an additional external B-field that contributes to the magnetic field detected by a sensor.

Many magnetic materials have a maximum magnetization, known as magnetic saturation, beyond which an increase in the applied magnetic field does not correspond to an increase in the magnetization of the material. Exploiting this property enables greater specificity to biological iron by measuring the harmonic frequencies that arise from magnetic saturation of the iron. In magnetic saturation methods, the applied magnetic field becomes strong enough that the magnetization resulting from the applied magnetic field is no longer linear. Nonlinear magnetization M as a function of H is typically modeled with a Langevin function:

$$L ( x ) X = coth ( x ) - \frac{1}{x} .$$

Regardless of choice of nonlinear function, a Taylor series expansion of the Langevin function

$$L ( x ) = \frac{1}{3} x - \frac{1}{45} {x}^{3} + …$$

may be used to approximate the magnetization in the nonlinear partially saturated regime. The applied magnetic field can then be substituted into the Taylor series to model the susceptibility behavior at AC frequencies. The AC susceptibility response of biological iron like ferritin and hemosiderin has been studied previously and displays characteristics of Neel relaxation nanoparticles with peak out of phase susceptibility in the tens of megahertz. A common method is to model the magnetically partially saturated regime of ferritin with a combined Langevin function and a linear term. At low field, the Langevin function fits the saturation characteristics but at high field the linear term fits the saturation characteristics. This method while not completely accurate has been shown to be a good fit with a more complete anisotropic magnetization model.

A simple model of the AC magnetic susceptibility of iron molecules is given by

$${χ}^{′} = {χ}_{0} \frac{1}{1 + {( ωτ )}^{2}}$$

$${χ}^{″} = {χ}_{0} \frac{ωτ}{1 + {( ωτ )}^{2}}$$

where χ′ and χ″ are the in-phase and out-of-phase magnetic susceptibility, χ₀ is the DC magnetic susceptibility, wis the frequency in radians and τ is a relaxation time constant. The relaxation time constant governs how fast a molecule will align with, and then relax back into, a random state in the presence of an external magnetic field. In general, small molecules have two types of relaxation, called Brownian relaxation and Néel relaxation. Brownian relaxation involves the entire particle rotating inside the magnetic field while Néel relaxation involves the magnetic domain rotating inside the magnetic field without molecular motion. The magnetic susceptibility of a molecule is a combination of these two relaxation types that depends on molecular size, temperature and the surrounding medium. The Néel relaxation time constant, τ, is given by the equation:

$$τ = \frac{\sqrt{π}}{2} {τ}_{0} \frac{exp ( \frac{κ ν}{{k}_{b} T} )}{\sqrt{\frac{κ ν}{{k}_{b} T}}}$$

where KV is an energy barrier related to the size of the molecules, τ₀ is a constant, k_{b} is Boltzmann's constant, and T is temperature. The equation shows that Néel relaxation is highly dependent on the size and temperature of the molecules.

Body iron stores primarily consist of hemosiderin and ferritin molecules that are micrometer to nanometer in size, allowing for a large Neel relaxation susceptibility at low temperature. Spectroscopic measurements of body iron improve at low temperature because responses to different applied magnetic field frequencies are larger than at body temperature. In addition, magnetic susceptibility exhibits nonlinear behavior as applied AC and DC magnetic fields are increased, particularly at magnetic fields less than 1 T. Thus, improved quantification of iron content is possible for ex vivo biological samples subjected to cryogenic temperatures.

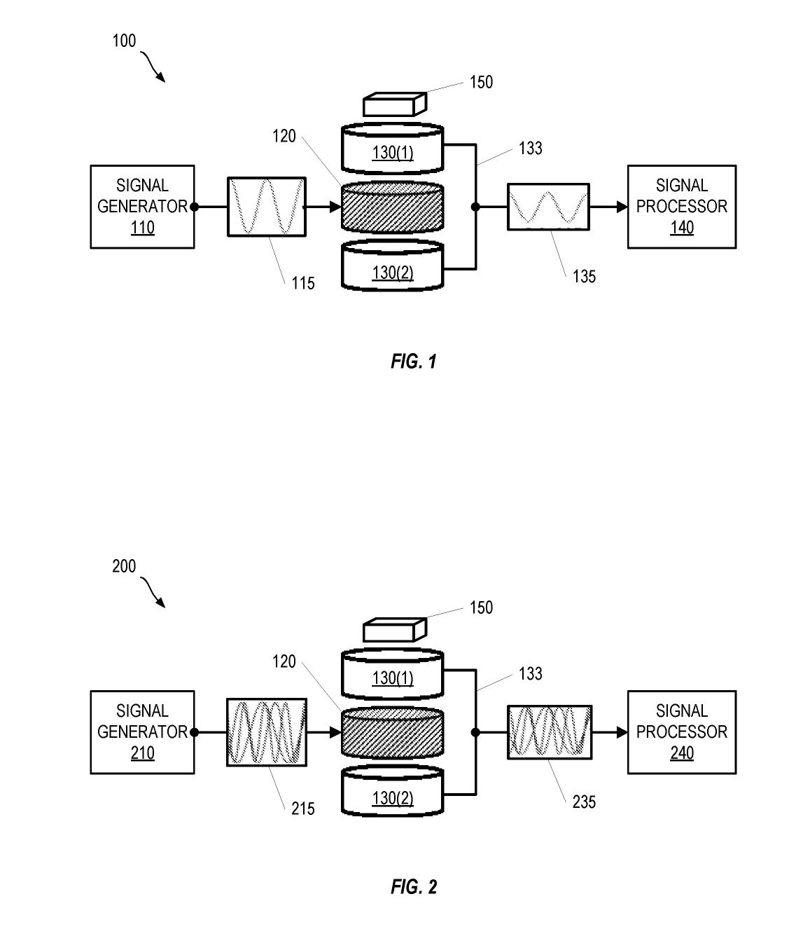

FIG. 1 is a schematic diagram showing an embodiment of a system for magnetic assessment of body iron stores 100. System 100 includes a signal generator 110 capable of generating alternating current (AC) signals, such as AC signal 115 for example. In an embodiment, signal generator 110 includes a computer (including software, processor and memory) used to produce a digitized signal, a digital-analog converter (DAC) array to convert the signal from digital to analog, and a multifunction digital acquisition (DAQ) device to control the DAC. Signal generator 110 is used to produce an AC signal 115 having significant components at one, two, or more frequencies between 100 Hz and 10 kHz. Signal generator 110 includes an amplifier to drive coil 120. In an embodiment, signal generator 110 includes two amplifiers, such as LME 49720 and LME 49610 from Texas Instruments (Dallas, Tex., USA), placed in series to create low noise, high power currents. The current in the coils depends on the testing frequency but ranges between 100 mA to 1 A for example.

Signal generator 110 is configured to generate AC signal 115 to drive a first excitation coil 120. First excitation coil 120 therefore generates an AC magnetic field at the same frequency as the AC signal of signal generator 110. First excitation coil 120 is, in one embodiment, a coil wound with 0.3 mm diameter wire, having inductance 7 mH, and resistance 11.8Ω at direct current (DC); and dimensions of 15 mm inner diameter, by 15 mm height, by 26 mm outer diameter, such as a Jantzen-1257 coil from Jantzen (Praestoe, Denmark). First excitation coil 120 is configured to preferentially excite nearby tissue containing iron and less than 1 cm into bone marrow, for example. In an embodiment, first excitation coil 120 produces magnetic fields less than 10 mT in tissue to ensure patient safety.

A detection coil 130 is an example of a sensor adapted to detect the AC magnetic field of first excitation coil 120. In an embodiment, detection coil 130 is also a Jantzen-1257 coil (same as excitation coil 120). As depicted in FIG. 1, detection coil 130 may include two detection coils 130(1), 130(2) configured to form a differential detection coil pair 133. Detection coil pair 133 may be arranged in parallel with first excitation coil 120 as shown in FIG. 1 or pair 133 may be arranged perpendicular to first excitation coil 120 (see FIG. 6). Differential detection coil pair 133 is configured to detect AC magnetic field differences from first excitation coil 120 as sensed at the first and second coil locations, and to generate a corresponding signal 135. Alternatively, other types of magnetic sensors may be used in place of differential detection coil pair 133 to detect the magnetic field generated by first excitation coil 120, such as a fluxgate magnetometer, magnetoresistive magnetometer, or Hall-effect magnetometer.

When a sample containing iron 150 is placed in proximity to system 100 nearer one detection coil than the other, for example nearer detection coil 130(1), iron in sample 150 perturbs the AC magnetic field generated by first excitation coil 120 at the nearer detection coil 130(1) more than at the more distant detection coil 130(2). In an embodiment, sample 150 is centered inside detection coil 130(1) and partially inside first excitation coil 120. Differential detection coil pair 133 detects the perturbed AC magnetic field by subtracting a signal in distant coil 130(2) from the near detection coil 130(1), and generates a corresponding difference signal 135 with corresponding perturbations.

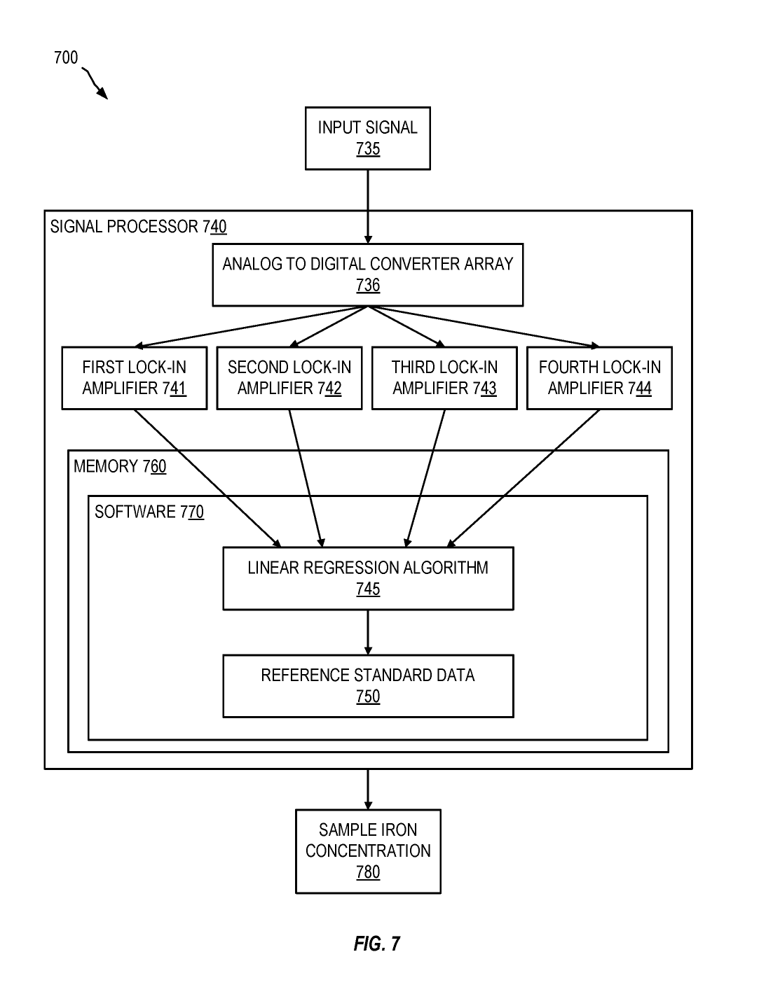

A signal processor 140 is configured to amplify and process signal 135. In an embodiment, signal processor 140 includes a multifunction DAQ device such as NI-USB 6289 from National Instruments (Austin, Tex., USA), which acquires signal 135 and converts it from analog to digital. In an embodiment, signal processor 140 includes a digital lock-in amplifier configured to acquire and amplify signal 135. In an embodiment, signal processor 140 includes a fourth order analog Butterworth filter with a cutoff frequency of 20 kHz to low-pass filter signal 135. An example showing details of signal processor 140 is shown in FIG. 7.

FIG. 2 is a schematic diagram showing one system for magnetic assessment of body iron stores 200, where system 200 is an embodiment of system 100 of FIG. 1. System 200 includes a signal generator 210 capable of generating AC signals at multiple frequencies, such as signal 215. Signal generator 210 is configured to drive first excitation coil 120 with multiple-frequency AC signal 215. First excitation coil 120 is configured to generate a multiple-frequency AC magnetic field according to signal 215 from signal generator 210. Differential detection coil pair 133 is configured to detect differences in magnetic fields from first excitation coil 120 at a first and second side of the excitation coil 120, and to generate a corresponding difference signal, such as a multiple-frequency signal 235 for example. When a sample containing iron 150 is placed in proximity to one side of system 200, iron in sample 150 perturbs the multiple-frequency AC magnetic field generated by first excitation coil 120. Differential detection coil pair 133 detects the perturbed multiple-frequency AC magnetic field and generates multiple-frequency signal 235 with corresponding perturbations. A signal processor 240 is configured to process multiple-frequency signal 235. In an embodiment, signal processor 240 includes a plurality of lock-in amplifiers configured such that each lock-in amplifier acquires at a different frequency. In an embodiment, signal processor 240 includes a linear regression algorithm for determining a collective effect of iron on multiple-frequency signal 235 (FIG. 7 shows example signal processor details).

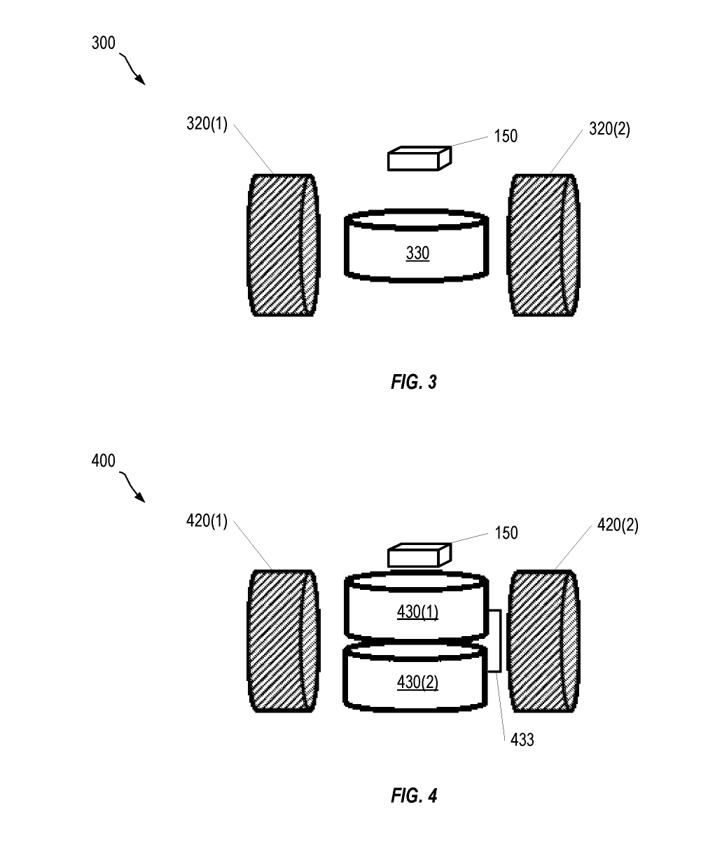

FIG. 3 shows an alternate orientation for excitation and detection coils 300 used in a system for magnetic assessment of body iron stores, such as system 200 of FIG. 2. Specifically, alternate orientation 300 shows a first excitation coil 320(1) aligned in parallel with a second excitation coil 320(2). Detection coil 330 is located between, and aligned perpendicular to, first excitation coil 320(1) and second excitation coil 320(2). Excitation coils 320(1), 320(2) are examples of excitation coil 120 of FIG. 1, and detection coil 330 is an example of detection coil 130(1) of FIG. 1. A perpendicular alignment between excitation coils 320(1), 320(2) and detection coil 330 reduces direct coupling between them and helps localize zones of high intermodulation frequency harmonics for non-linear responses. But, a higher magnetic field strength is required when excitation coils 320(1), 320(2) are aligned perpendicular to detection coil 330, compared to a parallel alignment of excitation and detection coils, such as those shown in FIGS. 1 and 2.

FIG. 4 shows an alternate orientation for excitation and detection coils 400 used in a system for magnetic assessment of body iron stores, such as system 200 of FIG. 2. Specifically, alternate orientation 400 shows a first detection coil 430(1) and a second detection coil 430(2) stacked in parallel with one another and configured to form a differential detection coil pair 433. Differential detection coil pair 433 is located between a first excitation coil 420(1) and a second excitation coil 420(2). Excitation coils 420(1), 420(2) are examples of excitation coil 120 of FIG. 1, and differential detection coil pair 433 is an example of differential detection coil pair 133 of FIG. 1. Compared to detection coil 330 of FIG. 3, detection coil 430(1) is positioned closer to the sample, such as sample 150 of FIG. 1. As in the arrangement of FIG. 3, the perpendicular alignment between the excitation and detection coils reduces direct coupling between them and helps localize zones of high intermodulation frequency harmonics for non-linear responses. But, a higher applied field strength is required when excitation coils 420(1), 420(2) are aligned perpendicular to detection coils 430(1), 430(2), compared to a parallel alignment of excitation and detection coils, such as those shown in FIGS. 1 and 2.

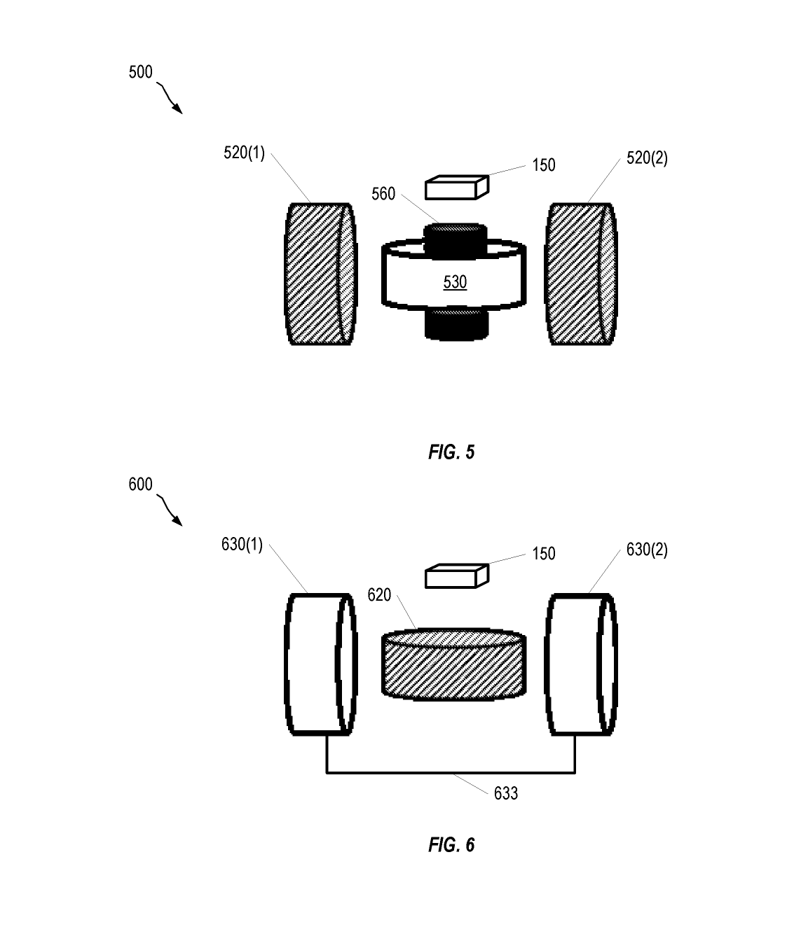

FIG. 5 shows an alternate orientation for excitation and detection coils 500 used in a system for magnetic assessment of body iron stores, such as system 200 of FIG. 2 Similar to alternate orientation 300 of FIG. 3, alternate orientation 500 shows a detection coil 530 located between a first excitation coil 520(1) and a second excitation coil 520(2), and aligned perpendicular to these two excitation coils. Excitation coils 520(1), 520(2) are examples of excitation coil 120 of FIG. 1, and detection coil 530 is an example of detection coil 130 of FIG. 1. Additionally, an embodiment of alternate orientation 500 includes a permanent DC magnet 560 located inside detection coil 530. Although FIG. 5 shows permanent DC magnet 560 located inside detection coil 530, other locations for magnet 560 near detection coil 530 are possible. Permanent DC magnet 560 produces a strong static non-linear magnetization of iron in the sample that may be used to bias the sample so that the AC stimulus generates even harmonic frequencies when nonlinearities in sample response—such as magnetic saturation—cause “clipping” and intermodulation products.

FIG. 6 shows an alternate orientation for excitation and detection coils 600 used in a system for magnetic assessment of body iron stores, such as system 200 of FIG. 2, in an embodiment. Specifically, alternate orientation 600 shows an excitation coil 620 located between, and aligned perpendicular to, a first detection coil 630(1) and a second detection coil 630(2) that are configured to form a differential detection coil pair 633. Excitation coil 620 and differential detection coil pair 633 are examples of excitation coil 120 and differential detection coil pair 133 of FIG. 1, respectively. Samples located close to excitation coil 620 primarily has a magnetized field perpendicular to detection coils 630(1), 630(2), while samples located deeper in tissue acts more like a dipole and has magnetized field lines that cut through the detectors. Therefore, alternate orientation 600 preferentially detects deeper iron sources compared to other orientations.

FIG. 7 is a diagram 700 showing input, output, and blocks of an embodiment of signal processor 740 used in a system for magnetic assessment of body iron stores, such as signal processor 240 of FIG. 2. An input signal 735 may include an AC signal from a detection coil, such as multiple-frequency AC signal 235 of FIG. 2. Signal processor 740 uses an analog to digital converter array 736 to accept input signal 735, convert it from analog to digital, and pass it to one or more lock-in amplifiers. In an embodiment, data is acquired at 100 kS/s with 18-bit analog to digital conversion.

Block diagram 700 of FIG. 7 shows four individual lock-in amplifiers: a first lock-in amplifier 711, a second lock-in amplifier 712, a third lock-in amplifier 713, and a fourth lock-in amplifier 714. However, signal processor 740 may include fewer than four or more than four individual lock-in amplifiers as appropriate for input signal 735. Each lock-in amplifier locks onto an individual frequency thereby separating a multiple-frequency signal into its individual frequency components. Signal processor 740 is for example a computer that includes a processor and memory 760. Memory 760 stores software 770 that includes machine readable instructions that when executed by processor 740 provide signal processing as described herein. Memory 760 and software 770 may be physically located in a self-contained device with other components of a system for magnetic assessment of body iron stores, such as system 200 of FIG. 2, or they may be located remotely as long as they may be electronically connected to the system. Software 770 includes a linear regression algorithm 745 and a set of reference-standard data 750. Linear regression algorithm 745 uses individual frequency components from lock-in amplifiers 741-744 to determine an effect of iron on a multiple-frequency AC signal. Using reference-standard data 750, software 770 correlates a result of linear regression algorithm 745 to a sample iron concentration 780. In an embodiment, the sample is biological. In particular embodiments, system 200 is placed over a suitable portion of an intact subject's anatomy, where magnetic fields penetrate the subject to reach and sense in vivo iron stores, in for example marrow of subject's sternum, liver, iliac crest, or vertebrae, and in juvenile subject's tibia or femur. Determined iron concentration of the patient's marrow or liver is used to determine patient's iron status. In alternative embodiments, the biological sample measured is ex vivo, such as a biopsy of bone marrow or liver, a bone marrow aspirate, or drawn blood. Ex vivo samples may be subjected to cryogenic temperatures for increasing the sensitivity of iron measurements, as described above.

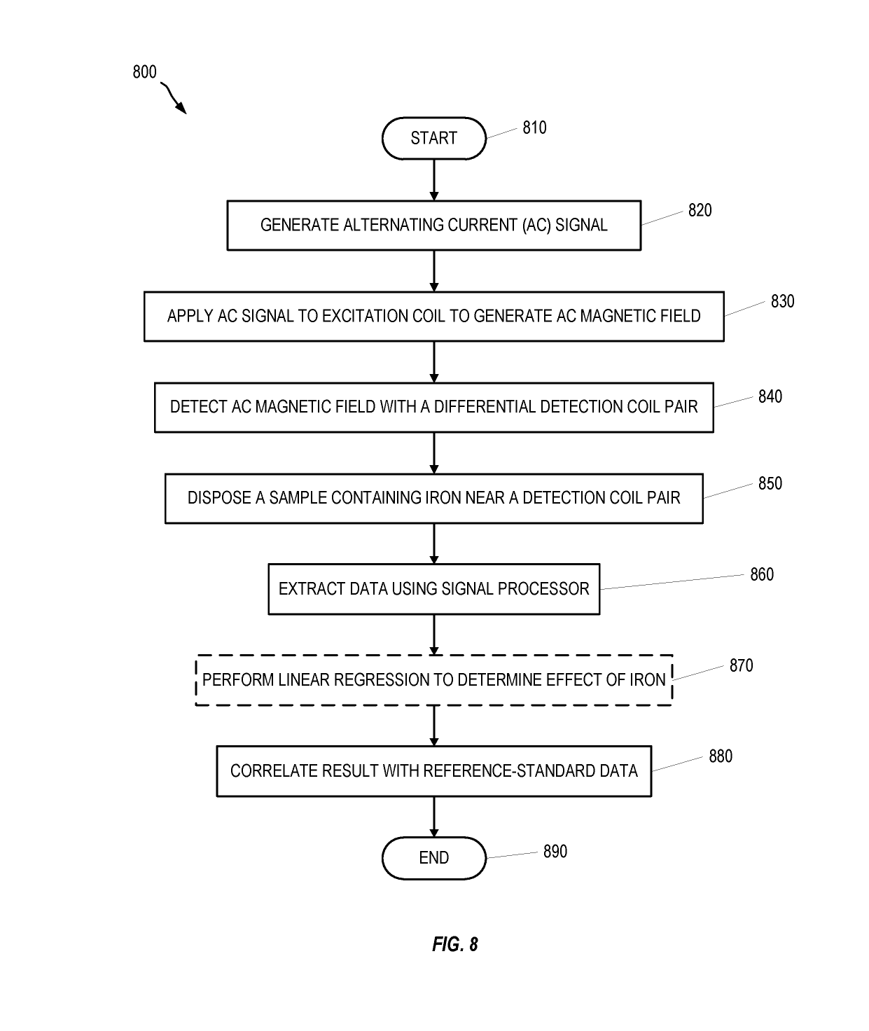

FIG. 8 is a block diagram showing steps of one method for magnetic assessment of body iron stores 800, in an embodiment. After starting 810, a signal generator 210 (FIG. 2) generates 820 AC signals such as multiple-frequency signal 215. The AC signal is applied 830 to excitation coil 220 to generate an AC magnetic field. The AC magnetic field is detected 840 with a differential detection coil pair 133. A sample containing iron is disposed 850 near a detection coil, such as detection coil 230(1) of FIG. 2, for example. Changes to the AC magnetic field caused by the sample containing iron are detected by differential detection coil pair 133, and data are extracted 860 using signal processor 240. A linear regression analysis is optionally performed 870 to determine an effect of iron in the sample using linear regression algorithm 745, if the applied signal is multiple-frequency. Otherwise, a single frequency signal does not require linear regression analysis. A result of the linear regression analysis is correlated 880 with reference-standard data 750 to determine an iron concentration of the sample. In an embodiment, the sample is a biological sample. In particular embodiments, system 200 is placed over a suitable portion of an intact subject's anatomy, where magnetic fields penetrate the subject to reach and sense iron stores in marrow of subject's sternum, liver, iliac crest, or vertebrae, and in juvenile subject's tibia, or femur. Iron concentration of the biological sample may be used to determine a patient's iron status.

### Examples of Use and Other Embodiments

Method 800 has several embodiments. The types of measurements made using embodiments of method 800 include proximity and spectroscopic measurements.

### Proximity Measurements

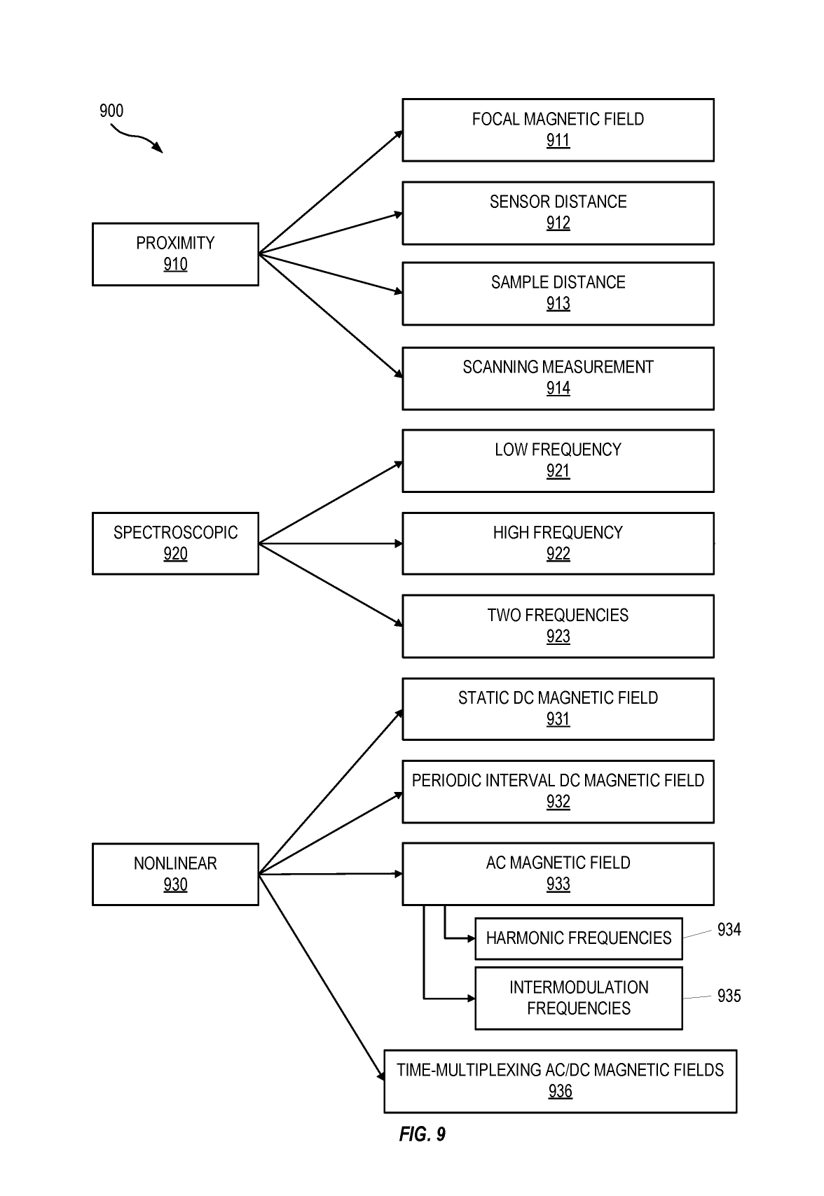

FIG. 9 shows an overview of different types of measurements and their embodiments. A first method to measure iron content is based on proximity of a sample or organism to a system for magnetic assessment of body iron stores, such as system 100 of FIG. 1 for example. For proximity methods 910, magnetization of biological iron remains in the linear magnetic susceptibility range at a given frequency.

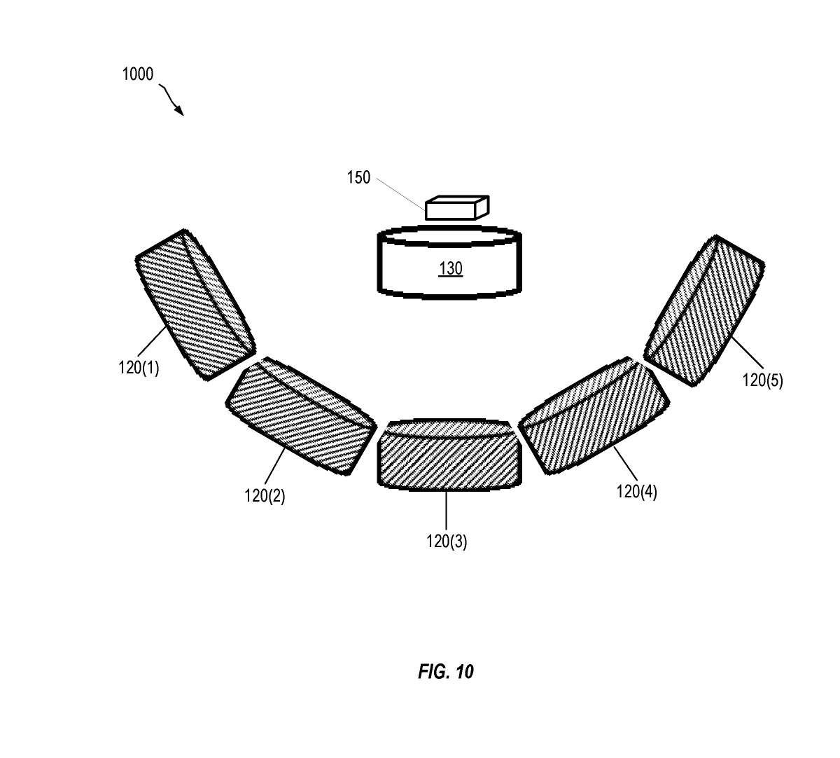

In a first embodiment, a focal magnetic field 911 generates a large magnetic field in sample 150, such as sternum, and a minimal magnetic field elsewhere, such as surrounding tissue. The resulting measurement primarily contains the magnetic susceptibility of bone marrow providing a measurement of iron stores. To create a focal magnetic field, two different techniques may be used. The first, shown in FIG. 10, uses a digitally controlled array of excitation coils 120 with a mathematical algorithm to create a magnetic field at a specific location and a minimal magnetic field elsewhere. FIG. 10 shows an embodiment with one detection coil 130 and five excitation coils 120 configured to create a focal magnetic field at the location of sample 150. A second and simpler technique uses a single excitation coil, such as first excitation coil 120 of FIG. 1, with a diameter sufficiently small to create a large local field a few millimeters from the sensor and a small magnetic field elsewhere.

In a second embodiment, proximity measurement 910 relies on a sensor distance measurement 912. In this example, measurements are performed at several distances from sample 150. This is accomplished by physically moving the sensor or by having a series of sensors positioned at varying distances from sample 150. Alternatively, sensor distance is held constant and magnetic field strength is varied to change an effective depth of magnetic field penetration. A series of measurements are acquired for various depths of interest. For example, a closest measurement includes sample 150 and other tissue, a second measurement primarily includes tissue, and a third measurement primarily contains no tissue. A mathematical model is used to determine bone marrow iron content from the series of measurements.

In a third embodiment, proximity measurement 910 is similar to sensor distance measurement 912 except sample distance is varied 913. Since sample 150 is not physically moved, a tissue like substance, such as a water bag, is used in its place. The water bag is placed between the skin and detection coil, such as detection coil 130(1) of FIG. 1, and a measurement is acquired. The water bag is then compressed such that detection coil 130(1) is moved closer to sample 150 and another measurement is acquired. Since water and tissue have nearly identical magnetic susceptibility, the difference in the two measurements is a function of the iron content of sample 150.

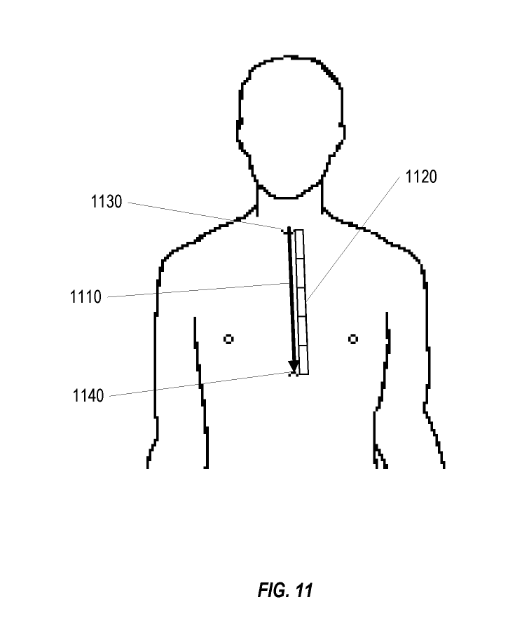

In a fourth embodiment, a scanning measurement 914 is performed by moving the detection coil across the sample at a constant distance from the sample. For example, detection coil 130(1) of FIG. 1 may be moved across sample 150 in a line scan. FIG. 11 shows an exemplary line scan 1110. Optionally, an alignment guide 1120, such as a plastic track or ruler, may be used for guiding the detection coil while performing a scan. Alternatively, system 100 may include a probe with an internal mechanism that moves the detection coil to perform the scan. Line scan 1110 may be performed along the sternum between anatomical landmarks, such as from a manubrium 1130 at the sternum top to a xiphoid process 1140 at the sternum bottom, as depicted in FIG. 11. Other line scan directions and/or patterns are possible without departing from the scope hereof. In an embodiment, scanning measurement 914 is performed by activating system 100 to initiate a scan by a user, such as by depressing a button, while the detection coil is located above manubrium 1130, the user then moves the detection coil along the sternum and deactivates the scan upon reaching xiphoid process 1140, such as by releasing the button. Alternatively, scanning measurement 914 is performed with a position tracker, such as an optical mouse/sensor or a rollerball tracking device, coupled with the detection coil for tracking position. Scanning measurement 914 may be used to measure additional tissues other than bone marrow for establishing background signals for improved measurements, including maximum and minimum AC magnetic field measurements.

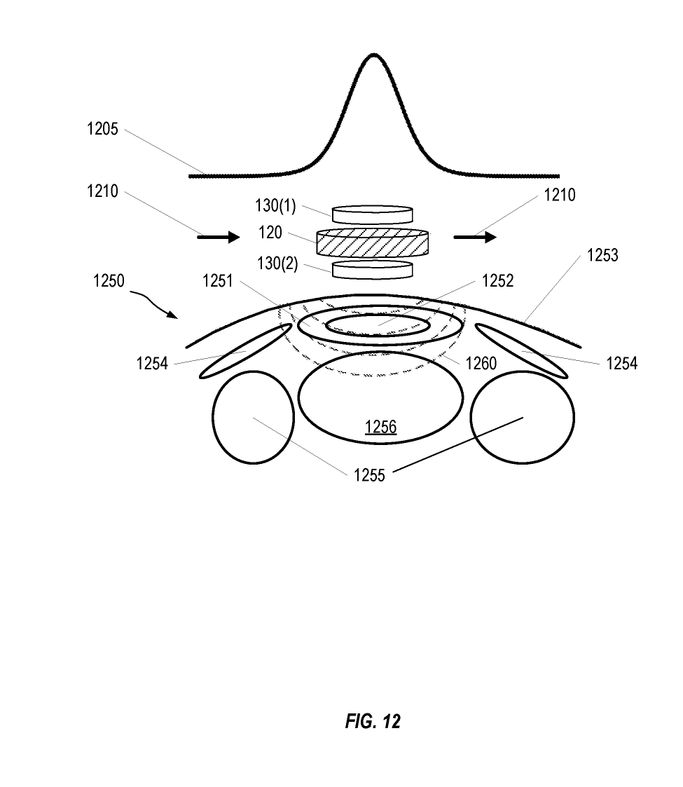

FIG. 12 illustrates a calculated sensitivity curve 1205 to iron during a line scan performed over a sternum 1251. In an embodiment, the line scan is performed by moving two detection coils 130(1), 130(2) configured to form a differential detection coil pair arranged in parallel with a first excitation coil 120, from FIG. 1, in the direction of arrows 1210, as shown in FIG. 12. Sternum 1251 having marrow 1252 is depicted within a cross-sectional representation of a chest 1250, including skin 1253, ribs 1254, lungs 1255, and a mediastinum 1256 including a heart. A gradient of applied magnetic field intensity is depicted with dashed contour lines 1260 to illustrate penetration of the magnetic field into sample 1250. The calculated sensitivity curve 1205 shows an expected signal that may be used to determine iron content for sternum bone marrow 1252. For example, data from the peak or maximum of sensitivity curve 1205 may be used to determine iron content by comparing with minimum portions of sensitivity curve 1205, which provide a background signal. Alternatively, relative position along the line scan is used as a covariate in a linear regression model that serves to convert the magnetic signals into iron assessments.

### Spectroscopic Measurements

A second way (see FIG. 9) to measure iron content is based on spectroscopic measurements 920 using a system for magnetic assessment of body iron stores, such as system 200 of FIG. 2, for example Spectroscopic measurements 920 require magnetic fields at different frequencies. The reason spectroscopic measurements 920 are effective is that biological iron and tissue have different relative magnetic susceptibilities at different frequencies.

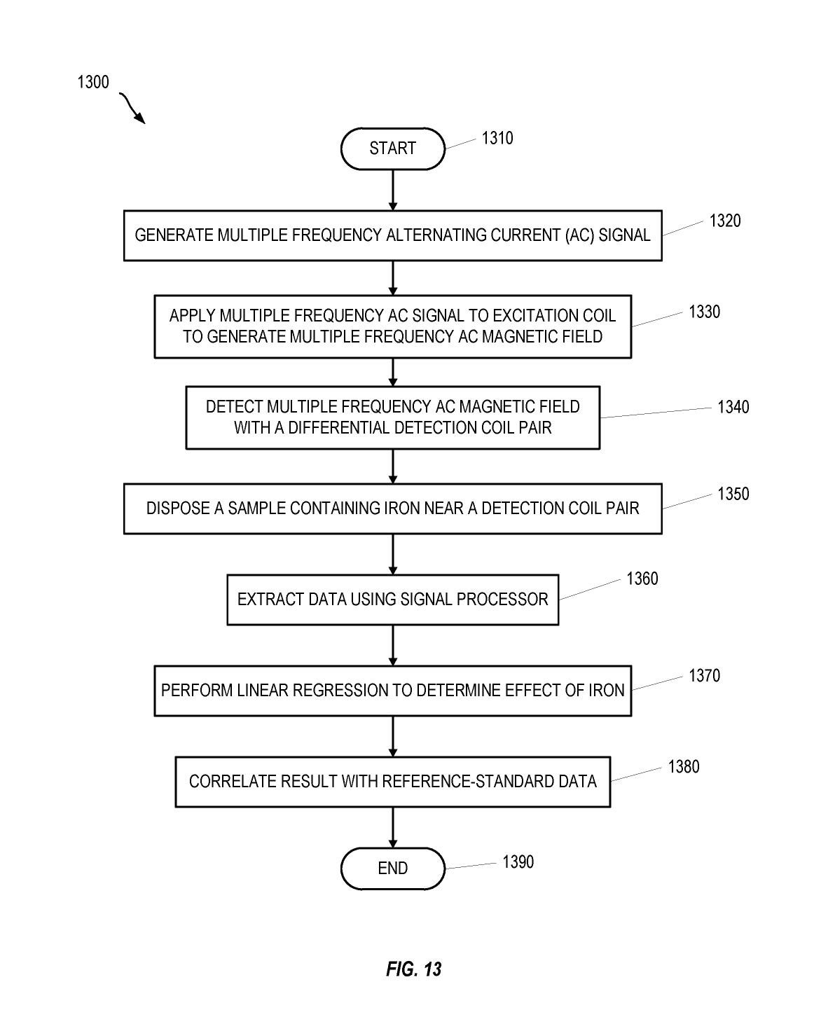

FIG. 13 is a block diagram showing steps of one embodiment of a method for magnetic assessment of body iron stores 1300 based on spectroscopic measurements 920. After starting 1310, a signal generator 210 (FIG. 2) generates 1320 multiple frequency AC signals such as signal 215. The AC signal is applied 1330 to excitation coil 220 to generate a multiple frequency AC magnetic field. The AC magnetic field is detected 1340 with a differential detection coil pair 133. A sample containing iron is disposed 1350 near a detection coil, such as detection coil 230(1) of FIG. 2, for example. Changes to the AC magnetic field caused by the sample containing iron are detected by differential detection coil pair 133, and the data are extracted 1360 using signal processor 240. A linear regression analysis is performed 1370 to determine an effect of iron in the sample using linear regression algorithm 745 (FIG. 7). A result of the linear regression analysis is correlated 1380 with reference-standard data 750 to determine an iron concentration of the sample.

In a first embodiment, spectroscopic measurements 920 (see FIG. 9) are performed at low frequencies 921, for example from 100 Hz to 1 kHz. In low frequency spectroscopy 921, a series of frequencies is chosen to separate the relative contributions of tissue and sample 150 iron content from the acquired measurements. Biological iron such as hemosiderin and ferritin behave like very small nanoparticles that undergo Néel relaxation. This results in a peak out-of-phase susceptibility response at several megahertz, well above the measurement range of a low frequency system. Due to the small Néel relaxation time constant, the separation of biological iron and tissue is more difficult to perform at low frequency. This is because the relative magnetic susceptibility of biological tissue and iron are more constant across the frequency space. However, since these frequencies are in the audio band, producing high quality magnetic fields is simplified.

In a second embodiment, spectroscopic measurements 920 are performed at high frequencies 922, for example from 10 kHz to 10 MHz. Due to the small relaxation times of biological iron species, a high frequency system is able to capture more magnetic susceptibility dynamics, enabling more separation between biological tissue and biological iron in a spectroscopic measurement. However, due to higher frequency measurements 922, the complexity of the magnetic field generation and measurement is increased.

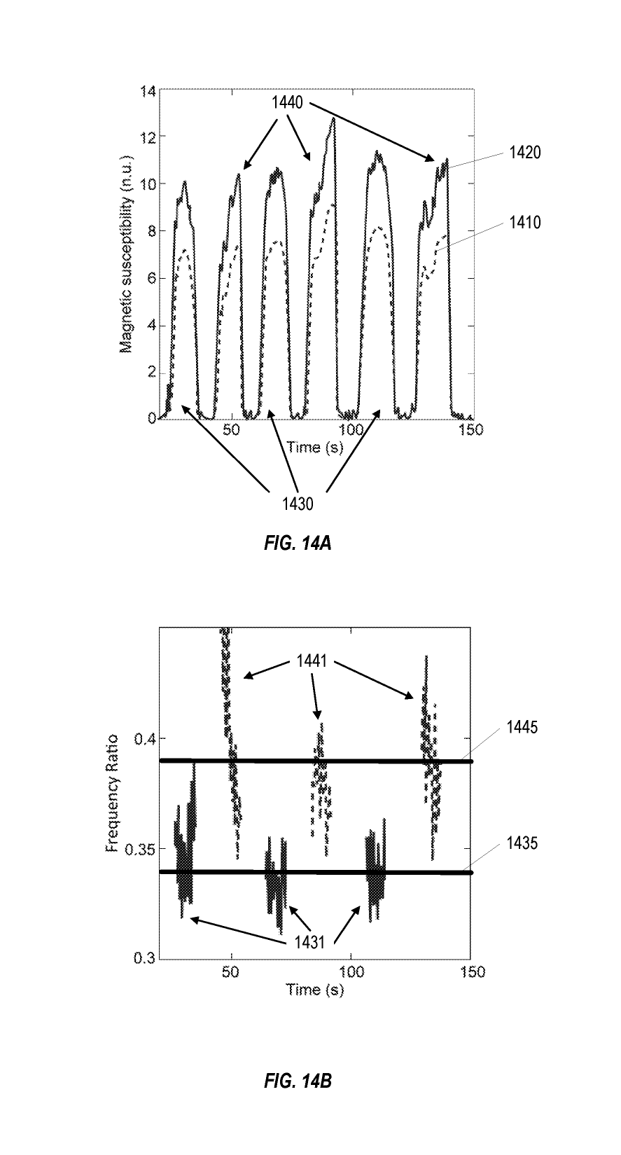

In a third embodiment, spectroscopic measurements 920 include two-frequency measurements 923. The two frequencies selected have a sufficiently large relative difference in magnetic susceptibility between biological tissue and biological iron. With a sufficient susceptibility difference, it is possible to separate biological tissue from biological iron from two measurements. In an embodiment, the two frequencies are used to calculate a frequency ratio of magnetic susceptibility. FIG. 14A plots magnetic susceptibility versus time for a series of measurements performed at a low frequency 1410 with dashed lines and an intermediate frequency 1420 with solid lines, with the two frequency measurements superimposed on each other. Low frequency 1410 is for example from 100 Hz to 1 kHz. Intermediate frequency 1420 is for example greater than 1 kHz and less than 10 kHz. In an embodiment, low frequency 1410 is about 200 Hz and intermediate frequency 1420 is about 2 kHz as shown in FIG. 14A. The measurement scans were alternately performed over a flank 1430 followed by a sternum 1440, each repeated twice. FIG. 14B depicts frequency ratios of the low frequency magnetic susceptibility divided by the intermediate frequency magnetic susceptibility from the data shown in FIG. 14A. Flank frequency ratios 1431 (solid lines) had a mean ratio 1435 of 0.34. Sternum frequency ratios 1441 (dashed lines) had a mean ratio 1445 of 0.39. The higher sternum mean ratio 1445 was expected due to a higher amount of iron in red bone marrow of the sternum.

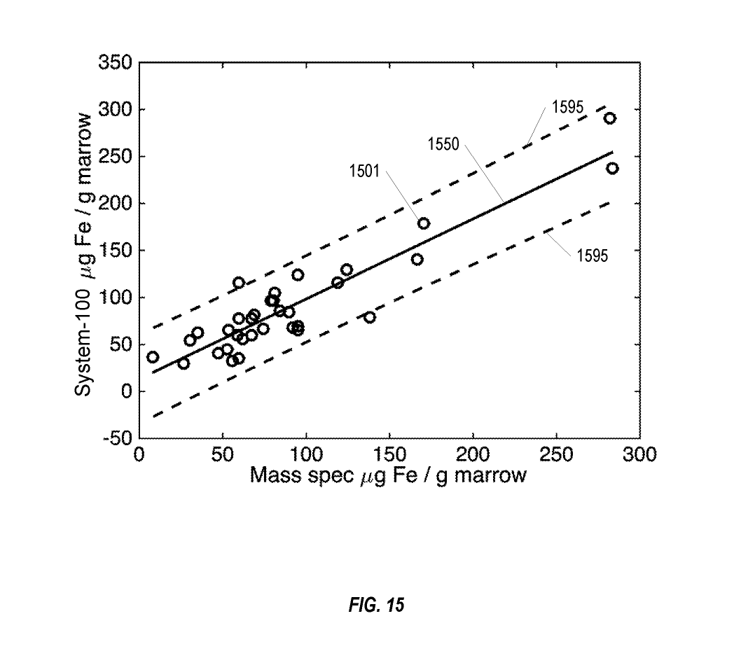

FIG. 15 illustrates exemplary results from two-frequency spectroscopic measurements 923 using system 100 (vertical axis) compared to mass spectrometry measurements (horizontal axis) for bone marrow samples, showing good agreement between the two methods. Specifically, values for thirty-three samples are represented with circles, such as circle 1501. The thirty-three values are statistically significantly correlated (R²=0.85, p<0.05) between the two methods, as shown with the solid trend line 1550 and the ninety-five percent confidence intervals 1595 shown with dashed lines.

### Nonlinear Measurements

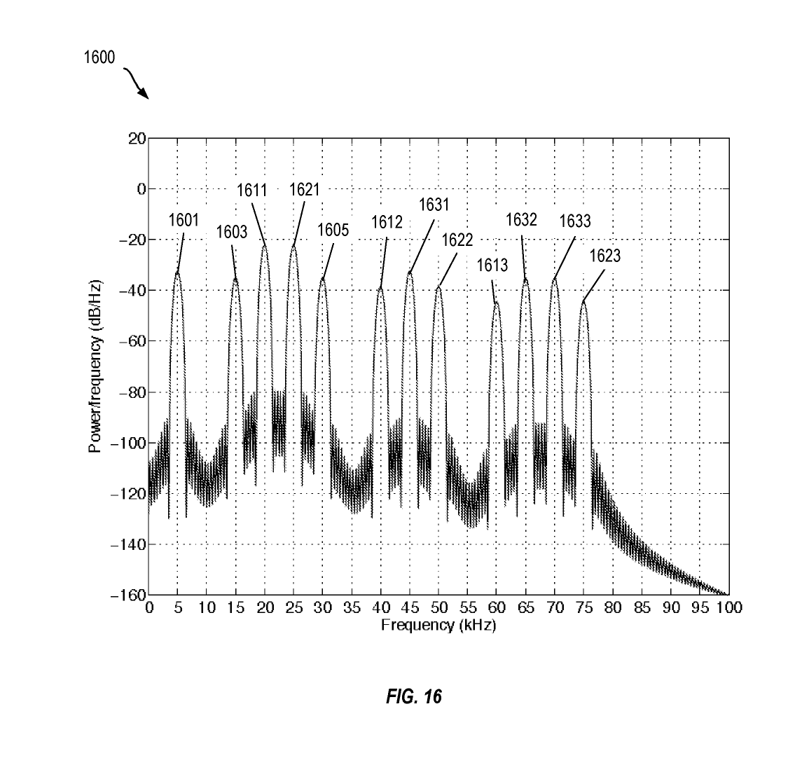

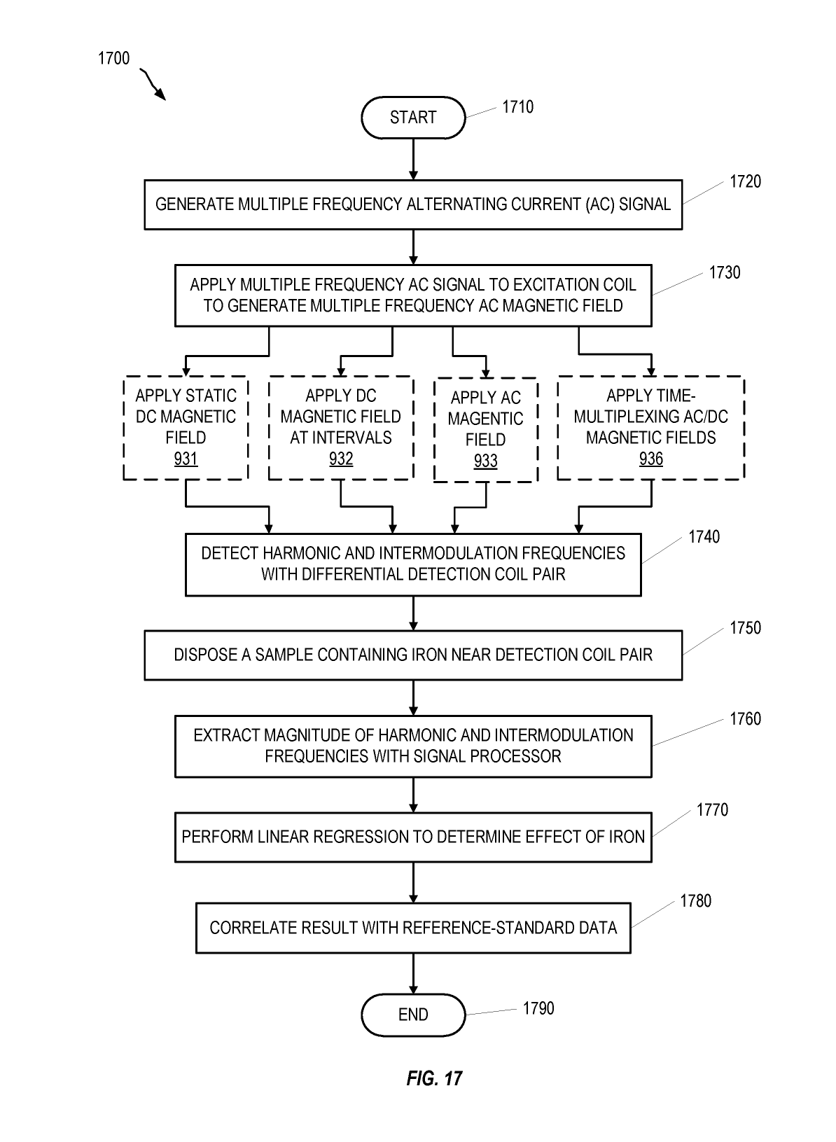

A third way (see FIG. 9) to measure iron content is based on nonlinear measurements 930 using a system for magnetic assessment of body iron stores, such as system 200 of FIG. 2, for example. To perform nonlinear measurements 930, large magnetic fields are generated in excitation coils (see FIG. 17 and description below), such as first excitation coil 120, thereby causing biological iron to enter a partially magnetically saturated regime. The resulting magnetization contains harmonic frequencies (see FIG. 16 and description below) Amplitude or phase shifting may be applied to generate a differential nonlinear response. Nonlinear measurements 930 of iron improve at low temperature because responses to different applied magnetic field frequencies are larger than at body temperature. For example, improved quantification of iron content is possible for ex vivo biological samples subjected to cryogenic temperatures.

FIG. 16 is a plot 1600 showing one embodiment of simulated harmonic and intermodulation frequencies resulting from nonlinearities in the response of sample 150. Peak 1611 represents a first fundamental frequency f₁ at 20 kHz, and peak 1621 represents a second fundamental frequency f₂ at 25 kHz. Second harmonic frequencies 2f₁ and 2f₂ are peak 1612 at 40 kHz and peak 1622 at 50 kHz, respectively. Peak 1631 shows a sum frequency of first and second fundamentals f₁+f₂ at 45 kHz, while peak 1601 shows a difference frequency between second and first fundamentals f₂−f₁ at 5 kHz. Difference frequencies between second and first fundamentals are 2f₁−f₂ at 15 kHz (peak 1603) and 2f₂−f₁ at 30 kHz (peak 1605). Sum frequencies between first and second fundamentals are 2f₁+f₂ at 65 kHz (peak 1632) and 2f₂+f₁ at 70 kHz (peak 1633). Third harmonic frequencies 3f₁ and 3f₂ are peak 1613 at 60 kHz and peak 1623 at 75 kHz, respectively. Additional harmonic frequencies (e.g., fourth harmonic, second sum) may also be present (not shown). Lock-in amplifiers are used to selectively measure the peaks. The magnitudes of the peaks are for example analyzed to determine iron content of sample 150.

In a first embodiment, nonlinear measurement 930 (see FIG. 9) includes a static DC magnetic field 931 applied to shift the AC magnetic field of first excitation coil 120 closer to a positive or negative saturation regime. The AC and DC magnetic fields are matched so that the AC magnetic field moves in and out of a measureable nonlinear portion of the iron magnetic saturation curve. Effectively, this limits the magnitude of the DC field.

In a second embodiment, nonlinear measurement 930 includes a DC magnetic field applied in periodic intervals 932 to shift the magnetization of the biological iron susceptibility response. This technique relies on the saturation characteristics of iron in which the susceptibility curve has different regions depending on the applied magnetic field. This helps further discriminate biological iron from other biological tissue. A variation of this embodiment involves switching the DC field from low to high in periodic intervals, which shifts the iron magnetization curve up and down and changes the harmonics produced. An advantage of this embodiment may include increased depth resolution.

In a third embodiment, nonlinear measurement 930 includes applying at least one AC frequency 933, which may be pulsed, to create harmonics due to nonlinearities in the sample. In this example, no DC magnetic field is applied. The resulting magnetization is symmetric and creates intermodulation products, such as harmonic 934 frequencies and intermodulation frequencies 935 (when two or more AC frequencies are applied). This simplified embodiment requires a stronger AC magnetic field to compensate for the absence of a DC field.

In a fourth embodiment, nonlinear measurement 930 involves time-multiplexing AC/DC magnetic fields 936, which includes applying a series of AC and optionally DC fields at different frequencies by turning on a first field pattern, then turning the first field pattern off, followed by turning on a second field pattern, and so on. Time-multiplexing AC/DC magnetic fields 936 allows sample measurement at variable AC/DC field strengths and frequencies sequentially instead of simultaneously. This technique is well suited for ex vivo samples such as those subjected to cryogenic temperatures because the amount of time required to perform the measurement may be longer than that of in vivo measurements.

FIG. 17 is a block diagram showing steps of one embodiment of a method for magnetic assessment of body iron stores 1700 based on nonlinear measurements. After starting 1710, a signal generator 210 (FIG. 2) generates 1720 multiple frequency AC signals such as signal 215. The AC signal is applied 1730 to excitation coil 220 to generate a multiple frequency AC magnetic field. In order to cause iron to enter a partial magnetic saturation regime, a large magnetic field is applied optionally as a DC magnetic field 931, a DC magnetic field applied at intervals 932, an AC magnetic field 933, or time-multiplexing AC/DC magnetic fields 936 (see FIG. 9). Harmonic 934 and intermodulation 935 frequencies (see e.g., FIG. 16) are detected 1740 with differential detection coil pair 133. Iron containing sample 150 is disposed 1750 near a detection coil, such as detection coil 230(1) of FIG. 2, for example Changes to the magnitude of harmonic and intermodulation frequencies caused by iron in sample 150 are detected by differential detection coil pair 133, and data are extracted 1760 using signal processor 240. A linear regression analysis is performed 1770 to determine an effect of iron in the sample using linear regression algorithm 745 (FIG. 7). A result of the linear regression analysis is correlated 1780 with reference-standard data 750 to determine an iron concentration of the sample.

### Combinations

The examples described above may be combined together to form a hybrid system. After initial screening of patients' bone marrow with magnetic assessment, determining width or volume of marrow channel may be desirable. Marrow channel width is observable with x-ray or ultrasound. Thus, combining magnetic assessment of bone marrow iron stores with a marrow width measurement may be used to further refine accuracy of the assessment.

Features described above as well as those claimed below may be combined in various ways without departing from the scope hereof. The following examples illustrate some possible, non-limiting combinations:

(A1) A system for magnetic assessment of body iron stores may include a first excitation coil adapted to generate a magnetic field, a signal generator configured to provide alternating current (AC) signals with a plurality of different frequencies to the first excitation coil that generates an AC magnetic field with a plurality of frequencies. The system may further include one or more sensors adapted to detect the AC magnetic fields, and a signal processor coupled to the one or more sensors and adapted to measure changes to the AC magnetic fields caused by proximity of the first excitation coil and the one or more sensors to iron.

(A2) The system denoted as (A1) may further include a position tracking device coupled with the one or more sensors and configured to track positions along an object as a plurality of AC magnetic field measurements are made while moving the one or more sensors along the object.

(A3) The system denoted as (A1) or (A2) may further include firmware in a processor configured to take a plurality of AC magnetic field measurements while moving the one or more sensors along the object, and a linear regression calculation of the plurality of AC magnetic field measurements is used to determine a region in the object of high iron concentration based upon the tracked positions.

(A4) In the system denoted as (A1) through (A3), the firmware may be configured to determine an iron concentration in a region of the object having higher concentration of iron than a background based on the plurality of AC magnetic field measurements and the tracked positions.

(A5) In the system denoted as (A1) through (A4), the one or more sensors may include a first detection coil and a second detection coil configured to form a differential detection coil pair.

(A6) In the system denoted as (A1) through (A5), the one or more sensors may be selected from the group consisting of a Hall effect magnetometer, a fluxgate magnetometer, and a magnetoresistive magnetometer.

(A7) In the system denoted as (A1) through (A6), the first and second detection coils may be located on opposite sides of, and aligned in parallel with, the first excitation coil.

(A8) In the system denoted as (A1) through (A7), the first detection coil and the second detection coil may be located on opposite sides of, and aligned perpendicular to, the first excitation coil.

(A9) The system denoted as (A1) through (A8) may further include a second excitation coil aligned in parallel with the first excitation coil and adapted to generate a magnetic field, and one or more sensors located between, and aligned perpendicular to, the first and second excitation coils.

(A10) In the system denoted as (A1) through (A9), the signal processor may include a multifunction data acquisition device, a plurality of lock-in amplifiers that each acquire an individual signal at a different frequency, and a linear regression algorithm for determining the effect of iron on a plurality of different frequency signals.

(A11) In the system denoted as (A1) through (A10), the object may be biological tissue, and the region having higher concentration of iron may be marrow.

(A12) In the system denoted as (A1) through (A11), the biological tissue may be selected from the group consisting of a sternum, a liver, an iliac crest, a vertebra, a tibia, and a femur.

(B1) A method of sensing iron concentrations in an object may include generating alternating current (AC) signals with a plurality of different frequencies, applying the AC signals to an excitation coil for generating a plurality of AC magnetic fields at different frequencies, detecting the AC magnetic fields with one or more magnetic sensors, disposing the object near one or more magnetic sensors such that iron in the object causes a change to the AC magnetic fields, and measuring changes to the AC magnetic fields with a signal processor.

(B2) The method denoted as (B1) including performing a scanning measurement by taking a plurality of AC magnetic field measurements while moving the one or more magnetic sensors along the object at a generally constant distance from the object. The method may further include a linear regression calculation of the plurality of AC magnetic field measurements that is used to determine a region in the object of high iron concentration based upon the tracked positions.

(B3) The method denoted as (B1) or (B2) including measuring a relative position of the one or more magnetic sensors during the scanning measurement, and using the relative position as a covariate in a linear regression model to convert magnetic sensor measurements into iron assessments.

(B4) In the method denoted as (B1) through (B3), the step of measuring changes to the AC magnetic fields may include acquiring signals using a plurality of lock-in amplifiers with each lock-in amplifier acquiring a different frequency signal, performing linear regression analysis on the plurality of different frequency signals to determine the effect of iron in the object, and determining an iron concentration in the object by correlating the result of the linear regression analysis to a set of reference-standard data.

(B5) In the method denoted as (B1) through (B4), the object may be an in vivo biological sample.

(B6) In the method denoted as (B1) through (B5), the in vivo biological sample may be selected from the group consisting of a sternum, a liver, an iliac crest, a vertebra, a tibia, and a femur.

(B7) In the method denoted as (B1) through (B6), the object may be an ex vivo biological sample subjected to cryogenic temperatures.

(B8) The method denoted as (B1) through (B7) including applying a static direct current (DC) magnetic field sufficient to partially magnetically saturate iron in the ex vivo biological sample to generate a non-linear response and harmonic frequencies.

(B9) The method denoted as (B1) through (B8) including generating a first AC magnetic field at a first frequency, generating a second AC magnetic field at a second frequency, such that the second AC magnetic field partially magnetically saturates iron in the ex vivo biological sample, and measuring intermodulation products such as harmonics at a third frequency, where the third frequency is not equal to the first or second frequency.

(B10) The method denoted as (B1) through (B9) including generating sequential patterns of AC magnetic fields at variable field strengths and frequencies.

(B11) The method denoted as (B1) through (B10) including generating sequential patterns of AC and DC magnetic fields at variable field strengths and frequencies.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A system for magnetic assessment of body iron stores, comprising:
a first excitation coil adapted to generate a magnetic field;
a signal generator configured to provide alternating current (AC) signals with a plurality of different frequencies to the first excitation coil, thereby generating an AC magnetic field with a plurality of frequencies;
one or more sensors adapted to detect the AC magnetic fields; and
a signal processor coupled to the one or more sensors and adapted to measure changes to the AC magnetic fields caused by proximity of the first excitation coil and the one or more sensors to iron.

2. The system of claim 1, further comprising a position tracking device coupled with the one or more sensors and configured to track positions along an object as a plurality of AC magnetic field measurements are made while moving the one or more sensors along the object.

3. The system of claim 2, further comprising firmware in a processor configured to take a plurality of AC magnetic field measurements while moving the one or more sensors along the object, wherein a linear regression calculation of the plurality of AC magnetic field measurements is used to determine a region in the object of high iron concentration based upon the tracked positions.

4. The system of claim 3, the firmware being configured to determine an iron concentration in a region of the object having higher concentration of iron than a background based on the plurality of AC magnetic field measurements and the tracked positions.

5. The system of claim 4, the one or more sensors comprising a first detection coil and a second detection coil configured to form a differential detection coil pair.

6. The system of claim 5, the one or more sensors being selected from the group consisting of a Hall effect magnetometer, a fluxgate magnetometer, and a magnetoresistive magnetometer.

7. The system of claim 5, the first and second detection coils, located on opposite sides of, and aligned in parallel with, the first excitation coil.

8. The system of claim 7, the first detection coil and the second detection coil being located on opposite sides of, and aligned perpendicular to, the first excitation coil.

9. The system of claim 8, further comprising:
a second excitation coil aligned in parallel with the first excitation coil and adapted to generate a magnetic field; and
one or more sensors located between, and aligned perpendicular to, the first and second excitation coils.

10. The system of any one of claim 1, the signal processor comprising:
a multifunction data acquisition device;
a plurality of lock-in amplifiers, wherein each lock-in amplifier acquires an individual signal at a different frequency; and
a linear regression algorithm for determining the effect of iron on a plurality of different frequency signals.

11. The system of claim 10, the object comprising in vivo biological tissue, and the region having higher concentration of iron being marrow.

12. The system of claim 11, the biological tissue comprising tissue selected from the group consisting of a sternum, a liver, an iliac crest, a vertebra, a tibia, and a femur.

13. A method of sensing iron concentrations in an object, comprising:
generating alternating current (AC) signals with a plurality of different frequencies;
applying the AC signals to an excitation coil for generating a plurality of AC magnetic fields at different frequencies;
detecting the AC magnetic fields with one or more magnetic sensors;
disposing the object near one or more magnetic sensors, wherein iron in the object causes a change to the AC magnetic fields; and
measuring changes to the AC magnetic fields with a signal processor.

14. The method of claim 13, further comprising performing a scanning measurement by taking a plurality of AC magnetic field measurements while moving the one or more magnetic sensors along the object at a generally constant distance from the object, wherein a linear regression calculation of the plurality of AC magnetic field measurements is used to determine a region in the object of high iron concentration.

15. The method of claim 14, further comprising measuring a relative position of the one or more magnetic sensors during the scanning measurement, and using the relative position as a covariate in a linear regression model to convert magnetic sensor measurements into iron assessments.

16. The method of claim 15, the step of measuring changes to the AC magnetic fields comprising:
acquiring signals using a plurality of lock-in amplifiers, wherein each lock-in amplifier acquires a different frequency signal;
performing linear regression analysis on the plurality of different frequency signals to determine the effect of iron in the object; and
determining an iron concentration in the object by correlating the result of the linear regression analysis to a set of reference-standard data.

17. The method of claim 16, the object comprising an in vivo biological sample.

18. The method of claim 17, the in vivo biological sample comprising tissue selected from the group consisting of a sternum, a liver, an iliac crest, a vertebra, a tibia, and a femur.

19. The method of claim 16, the object comprising an ex vivo biological sample subjected to cryogenic temperatures.

20. The method of claim 19, further comprising applying a static direct current (DC) magnetic field sufficient to partially magnetically saturate iron in the ex vivo biological sample to generate a non-linear response and harmonic frequencies.

21. The method of claim 20, comprising:
generating a first AC magnetic field at a first frequency;
generating a second AC magnetic field at a second frequency, wherein the second AC magnetic field partially magnetically saturates iron in the ex vivo biological sample; and
measuring intermodulation products such as harmonics at a third frequency, wherein the third frequency is not equal to the first or second frequency.

22. The method of claim 21, comprising generating sequential patterns of AC magnetic fields at variable field strengths and frequencies.

23. The method of claim 22, comprising generating sequential patterns of AC and DC magnetic fields at variable field strengths and frequencies.

24. The system of claim 13, the one or more sensors comprising a first detection coil and a second detection coil configured to form a differential detection coil pair.

25. The system of claim 24, the first and second detection coils, located on opposite sides of, and aligned in parallel with, the first excitation coil.

26. The system of claim 24, the first detection coil and the second detection coil being located on opposite sides of, and aligned perpendicular to, the first excitation coil.
